(19) United States
(12) Patent Application Publication
(10) Pub. No.: US 2011/0054269 A1
(43) Pub. Date: Mar. 3, 2011

(54) METHOD AND APPARATUS FOR PROVIDING AND SHARING COMPREHENSIVE HEALTH INFORMATION

(75) Inventors: Jae-young Lee, Hwaseong-si (KR); Hyung-jin Seo, Hwaseong-si (KR); Sung-hyun Cho, Seoul (KR); Cory Kim, Hwaseong-si (KR); Jong-eun Yang, Seoul (KR)

(73) Assignee: Samsung Electronics Co., Ltd., Suwon-si (KR)

(21) Appl. No.: 12/869,374

(22) Filed: Aug. 26, 2010

(30) Foreign Application Priority Data
Aug. 26, 2009 (KR) 10-2009-0079191

(51) Int. Cl. A61B 5/00

(52) U.S. Cl. 600/300

(57) ABSTRACT

A method and apparatus for providing life health information is provided. The method includes obtaining life health information based on at least one of a physical health state of a person, a psychological health state of the person, and interactive exchange information between the person and other people; setting a personal metaphor that visualizes the life health information of the person; and displaying a visualized image corresponding to the personal metaphor.

### PRIORITY

This application claims priority to Korean Patent Application No. 10-2009-0079191, filed on Aug. 26, 2009, in the Korean Intellectual Property Office, the disclosure of which is incorporated herein in its entirety by reference.

### BACKGROUND OF THE INVENTION

1. Field of the Invention

The present invention relates to a method and apparatus for providing and sharing comprehensive health information.

2. Description of the Related Art

Due to developments in technology and in equipment, physical exercise is becoming easier for people, but many people lack the time to take care of their health because of their busy lifestyles. Also, it is often not enough just to maintain one's physical health in today's increasingly complex modern society.

Various technologies for measuring the physical health of a person are developed in conjunction with medical science but it is difficult for the general public to understand the technical knowledge needed to understand medical health indexes.

### SUMMARY OF THE INVENTION

The present invention provides a method and apparatus for providing and sharing comprehensive health information about a person and other people.

According to an aspect of the present invention, there is provided a method of providing life health information, the method including obtaining life health information based on at least one of a physical health state of a person, a psychological health state of the person, and interactive exchange information between the person and other people; setting a personal metaphor that visualizes the life health information of the person; and displaying a visualized image corresponding to the personal metaphor.

The method may further include storing a recommended life guiding plan that may be provided to the person, corresponding to the life health information of the person.

The method may further include sharing life health information of group members between the group members by transmitting visualized images corresponding to personal metaphors of the group members.

The method may further include providing information about a recommended life guiding plan to the person based on the obtained life health information of the person.

The method may further include updating information about the recommended life guiding plan.

According to another aspect of the present invention, there is provided a life health information providing apparatus including a personal life health information obtaining unit obtaining life health information based on at least one of a physical health state of a person, a psychological health state of the person, and interactive exchange information between the person and other people; a metaphor setting unit setting a personal metaphor that visualizes the life health information of the person; and a display unit displaying a visualized image corresponding to the personal metaphor.

The life health information providing apparatus may further include a life guiding plan database that stores information about a recommended life guiding plan that is to be provided to the person based on the life health information of the person; and a recommended life guiding plan selecting unit that selects information about the recommended life guiding plan for the person based on the obtained life health information of the person, wherein the display unit displays the information about the selected recommended life guiding plan.

According to another aspect of the present invention, there is provided a computer readable recording medium having embodied thereon a program for executing the method described above.

### BRIEF DESCRIPTION OF THE DRAWINGS

The above and other features and advantages of the present invention will become more apparent by describing in detail exemplary embodiments thereof with reference to the attached drawings in which:

FIG. 1A is a block diagram illustrating a life health information providing apparatus according to an embodiment of the present invention;

FIG. 1B is a block diagram illustrating a life health information providing apparatus according to another embodiment of the present invention;

FIG. 2A illustrates a metaphorical image that visualizes life health information of a user, according to an embodiment of the present invention;

FIG. 2B illustrates metaphorical images that visualize life health information of family members, according to an embodiment of the present invention;

FIG. 3 illustrates metaphorical images of life health information of group members, according to another embodiment of the present invention;

FIG. 4 illustrates a method of sharing life health information of family members, according to an embodiment of the present invention;

FIGS. 5A and 5B illustrate screens providing recommended life guiding plans with respect to life health information of the user, according to an embodiment of the present invention;

FIG. 6 illustrates a screen providing a recommended life guiding plan with respect to life health information of other people, according to an embodiment of the present invention;

FIG. 7 is a flowchart illustrating a method of providing life health information, according to an embodiment of the present invention; and

FIG. 8 is a flowchart illustrating a method of providing life health information, according to another embodiment of the present invention.

### DETAILED DESCRIPTION OF THE INVENTION

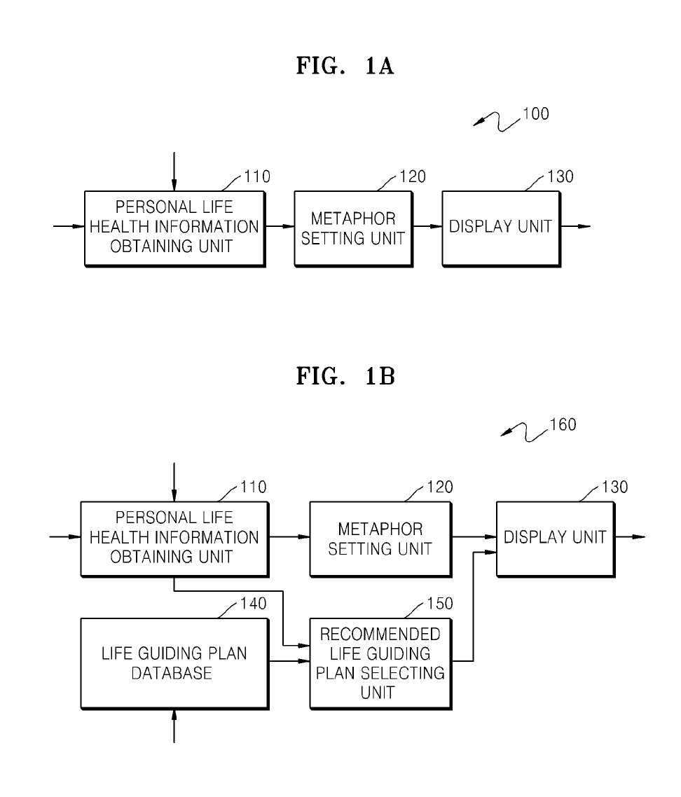

Hereinafter, a method and apparatus for providing and sharing life health information of a person and other people according to embodiments of the present invention will be described with reference to FIGS. 1A through 8.

FIG. 1A is a block diagram illustrating a life health information providing apparatus 100 according to an embodiment of the present invention.

The life health information providing apparatus 100 includes a personal life health information obtaining unit 110, a metaphor setting unit 120, and a display unit 130.

The personal life health information obtaining unit 110 obtains life health information of a person. Life health refers to a state in which a person can live a free and happy life that is determined in consideration of not only a personal physical health index but also psychological states and personal social relationships. Social health refers to a state in which there is excellent interactive exchange between a person and other people. The life health information providing apparatus 100 may measure a social health state based on interactive exchange information between the person and the other people.

Accordingly, the personal life health information obtaining unit 110 obtains life health information based on a physical health state of a person, a psychological health state of the person, and interactive exchange information between the person and the other people, in total or separately.

In addition to the user, the life health information providing apparatus 100 may also manage life health information of other members of a predetermined group to which the user belongs. Life health information of the other people may be input to various health index measuring devices that are connected to the life health information providing apparatus 100 or may be received via a network.

The personal life health information obtaining unit 110 may obtain personal log information regarding communications between the person and the other people which is stored in a personal communication device as life health state information based on interactive exchange information between the person and the other people. The personal log information may include personal log information including communications from the person to the other people and personal log information including communications from the other people to the person.

Examples of the personal log information include log information related to email transmission and reception log information related to telephone transmission and reception, log information related to transmission and reception of text messages, and log information related to chatting between a person and the other people via a personal communication device.

Also, when a personal schedule may be managed using a personal communication device or a personal portable device and information about people to meet may be stored by using a schedule managing function of the device, information about appointments with the other people in schedule data of the person may be used as the interactive exchange information between the person and the other people.

In addition, information about a frequency of photographing the other people during a recent predetermined period of time may be used as life health state information based on interactive exchange information between the person and the other people using a communication device having a photographing function.

Also, various information regarding contexts between the person and the other people may be used as life health state information based on interactive exchange information between the person and the other people.

The personal life health information obtaining unit 110 may obtain information about a physical health index that is measured by using a device for measuring a physical health index of a person as life health state information based on a physical health state of the person. Examples of the physical health index may be various body conditions and health indexes of a person such as exercise, blood pressure, blood sugar level, heart rate, body temperature, obesity level, etc.

The personal life health information obtaining unit 110 may obtain resultant information that is measured using a device for measuring psychological stability of the person as life health state information based on a psychological health state. Psychological stability may be estimated by considering personal stress, emotional instability, a state of tension, or the like.

The device for measuring a physical health index or psychological stability may be connected to the life health information providing apparatus 100 via wires or in a wireless manner, via a network.

The life health information providing apparatus 100 obtains personal life health information and outputs the same to the metaphor setting unit 120.

The metaphor setting unit 120 sets a personal metaphor that visualizes personal life health information. A “metaphor” refers to an object that is designed to transfer a certain concept or information easily and quickly by inducing association of the user with a certain subject. The metaphor setting unit 120 may set a metaphor that may integrate a personal physical health state, a personal psychological health state, or interactive exchange information between the person and the other people and intuitively show the information.

The metaphor setting unit 120 may set a personal metaphor with respect to life health information based on the interactive exchange information between the person and the other people by referring to the exchanges between the person and the other people. Social exchange between the person and the other people may include repeated communications from the person to the other people or from the other people to the person.

However, degrees of communications from the person to the other people and from the other people to the person are not always the same. Accordingly, to determine a social health state of the person based on the social exchange between the person and the other people, degrees of communications between the person and the other people may be considered in total, or the degrees of communications from the person to the other people and the degree of communications from the other people to the person may be considered separately.

Furthermore, to assess the social exchange between the person and other people, a weighting factor may be added according to a communication method for the communications between the person and the other people. For example, log information related to transmission and reception of text messages via a mobile phone, log information related to telephone transmission and reception, log information related to email transmission and reception between a person and other people, log information related to chatting, schedule data about contacts with the other people, metadata of photographs of the other people that are photographed directly by the person, etc. may be used to assess the degree of social exchange between the person and the other people.

For example, when assessing the degree of social exchange between the person and other people, a high weight may be added to the schedule data about contacts with other people that is generated based on face-to-face meetings between the person and other people and metadata of photographs of the other people that are taken directly by the person. Also, a high weight may be added to communication methods that require more effort and time among on-line communication methods that include text messages, telephone conversations, e-mails, or chatting using a mobile phone. Also, the degree of social exchange may be assessed by considering the time or frequency of the communications.

The metaphor setting unit 120 may set a metaphor corresponding to personal life health information so as to include a first sub-metaphor indicating a physical health state of a person, a second sub-metaphor indicating a psychological health state of the person, and a third sub-metaphor indicating interactive exchange information between the person and the other people.

Alternatively, the metaphor setting unit 120 may separately set a metaphor indicating a physical health state of a person, a metaphor indicating a psychological health state of the person, and a metaphor indicating interactive exchange information between the person and the other people, to be expressed individually.

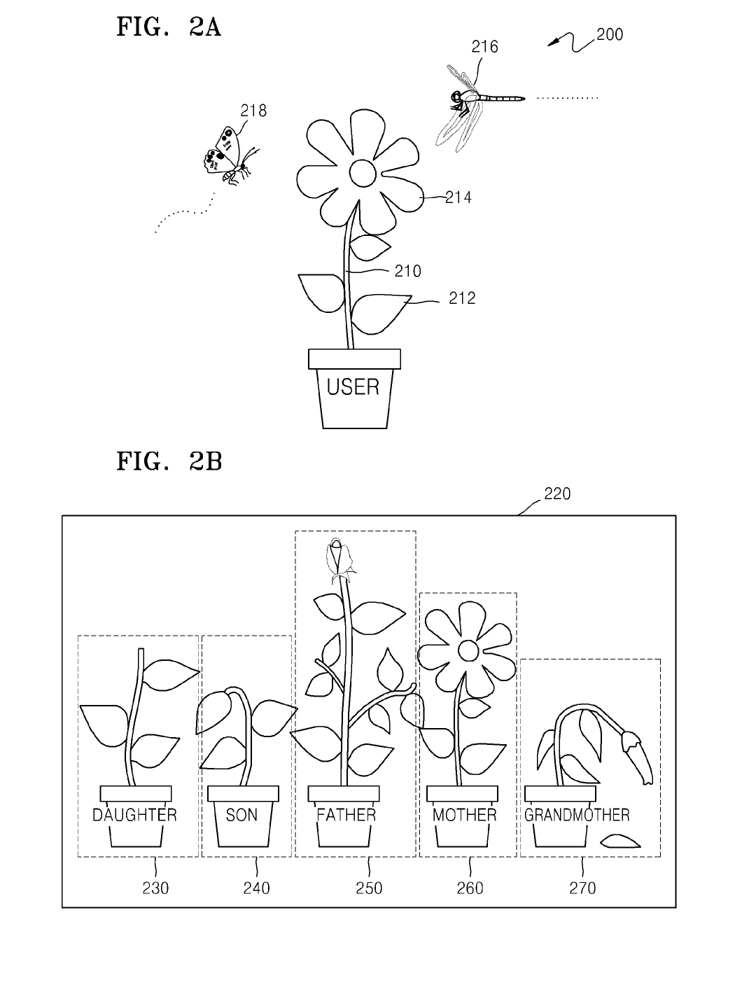

For example, as described in detail below with respect to FIG. 2A, the metaphor setting unit 120 may set a flower contained in a flowerpot, including a stem, a flower, a leaf, etc. as a metaphor corresponding to life health information of a person. In this case, the physical health state of the person may be expressed using the stem and the leaf, the psychological health state may be expressed using the flower, and the social health state may be expressed using butterflies or bees so that sub-metaphors corresponding to the physical health state, the psychological health state, and the interactive exchange information between the person and the other people are included in one metaphor corresponding to the life health information of the person.

In addition, as an alternative, the metaphor setting unit 120 may separately set a first flower in a first flowerpot representing a physical health state of a person, a second flower in a second flowerpot representing a psychological health state of the person, and a third flower in a third flowerpot representing interactive exchange information between the person and the other people.

Here, a flower in a flowerpot is used as an example of a metaphor for convenience of description. Also, the metaphor setting unit 120 may express personal life health information using ordinary images that the user may intuitively understand and recognize. For example, an animal, the sky, or weather may be used as a metaphor.

For example, when the metaphor setting unit 120 sets an animal as a metaphor corresponding to personal life health information, the size of the animal may be set to indicate the physical health state, degree of activity of the animal may be set to indicate the psychological health state, and sounds made by the animal may be set to indicate the interactive exchange information between the person and other people.

Alternatively, as another example, when the metaphor setting unit 120 sets a car as a metaphor corresponding to personal life health information, the degree of cleanliness of the car may indicate the physical health state, the engine sound of the car may indicate the psychological health state, and the degree of upgrading conditions of the car may indicate the interactive exchange information between the person and the other people.

As another alternative, when the metaphor setting unit 120 sets the sky including a sun, a cloud, and a bird as a metaphor corresponding to personal life health information, the sun may indicate the physical health state, the cloud may indicate the psychological health state, and the bird may indicate the interactive exchange information between the person and the other people.

As a further alternative, when the metaphor setting unit 120 sets weather as a metaphor corresponding to personal life health information, temperature and brightness may indicate the physical health state, rainfall and a thunderstorm may indicate the psychological health state, and wind speed may indicate the interactive exchange information between the person and the other people.

The metaphor setting unit 120 may also set a metaphor with respect to life health information of various individuals. Accordingly, when life health information of the user and other members of a predetermined group is input, metaphors may be set with respect to each of the individuals.

The display unit 130 displays a visualized image corresponding to personal metaphors. When metaphors are set with respect to life health information of the user and the other members by using the metaphor setting unit 120, all visualized images corresponding to the personal metaphors may be displayed. Accordingly, the user and the members may recognize life health information with respect to all of the members through the metaphorical visualized images.

According to the life health information providing apparatus 100, health information is provided by using a metaphor, and thus the user may easily and intuitively understand life health information of himself/herself or group members without expert knowledge about health information. Also, as life health information based on a social health state based on communications between the person and other people is provided using the life health information providing apparatus 100, the person may control both his/her physical health and psychological health and may be able to act in a healthy manner in social environments.

In addition, conventional metaphors representing health are used as application services through visualization of indicators of physical health usually from a first-person viewpoint. However, according to the life health information providing apparatus 100 according to the current embodiment of the present invention, life health information, to which the degree of interactive exchange between the user and another party is reflected, is used. Accordingly, in an application service method using metaphor markings according to the life health information providing apparatus 100, indexes from a second-person viewpoint may also be used.

That is, according to the life health information providing apparatus 100, health information of a group member is not shared with the same contents with everybody but is shown in regard to the relationship between the user and the group members. For example, regarding a group such as a family, a metaphor indicating the health of a daughter seen from the viewpoint of a father and a metaphor indicating the same from the viewpoint of a mother may be different.

Accordingly, according to the life health information providing apparatus 100, unlike conventional services that express only physical health information from a first-person viewpoint, metaphors indicating physical health information and psychological/emotional health information, and social health information may also be expressed from a second-person viewpoint and application services using the metaphors may be provided.

FIG. 1B is a block diagram illustrating a life health information providing apparatus 160 according to another embodiment of the present invention.

In addition to the personal life health information obtaining unit 110, the metaphor setting unit 120, and the display unit 130 of the life health information providing apparatus 100, the life health information providing apparatus 160 further includes a life guiding plan database 140 and a recommended life guiding plan selecting unit 150.

The life guiding plan database 140 stores information about recommended life guiding plans that may be provided to the user according to at least one of the physical health information and the psychological/emotional health information, and the social health information by using the life health information providing apparatus 160.

The life guiding plans may include information about physical lifestyle habits for physical health such as food menus, exercising methods, and sleeping times of a person. The life guiding plans may include hobbies such as listening to music, watching movies or travelling and stress relieving methods, and information about relieving psychological states. Also, the life guiding plans may include information about social exchange activities for social health including methods of contacting other people and activities that may be shared with the other people.

The information about the life guiding plans of the life guiding plan database 140 may be updated via a network or an external storage medium.

The recommended life guiding plan selecting unit 150 selects information about a life guiding plan that may be recommended to the person from the life guiding plan database 140 based on life health information of the person input by using the personal life guiding information obtaining unit 110. Information about the selected recommended life guiding plan may be output to the display unit 130 and displayed thereon.

The recommended life guiding plan selecting unit 150 may select a life guiding plan needed to improve a person's life and health, based on life health information of the person. Mapping of the life health information onto the life guiding plan may be determined using, for example, a mapping table that is stored in advance in the life health information providing apparatus 160.

Alternatively, information about the life guiding plan stored in the life guiding plan database 140 may include an index in regard to corresponding current life health information of the person. Accordingly, the recommended life guiding plan selecting unit 150 may search for information about life guiding plans including an index corresponding to the current life health information of the person and may select the same.

Also, the recommended life guiding plan selecting unit 150 may include a collection of algorithms for selecting life guiding plans according to life health information of a person, included in a software module or a hardware module.

Considering physical health states among the life health information of the person, the recommended life guiding plan selecting unit 150 may select life guiding plans for healthy physical lifestyle habits such as diets, exercising methods, or sleeping times of the person.

The recommended life guiding plan selecting unit 150 may select a life guiding plan for relieving psychological states, including hobbies and stress relieving methods such as listening to music, watching movies or travelling, in consideration of psychological health states among the life health information of the person.

The recommended life guiding plan selecting unit 150 may select a life guiding plan including social exchange activities for social health, including methods of contacting other people and activities that may be shared with the other people in consideration of interactive exchange information between the person and the other people among the life health information of the person.

The recommended life guiding plan selecting unit 150 may also select a recommended life guiding plan in consideration of personal characteristics such as gender, age, or preference of the person or other people.

The life health information providing apparatus 100 or 160 according to the embodiments of the present invention transmits visualized metaphorical image data corresponding to personal metaphors of group members to the group members, and thus the group members may share life health information of the group members and life guiding plans for the group members.

The life health information providing apparatus 160 provides a life guiding plan in consideration of other people based on life health information of the person so that the person may conveniently control his or her life and health. Also, the life health information providing apparatus 160 provides a life guiding plan and a social exchange plan based on life health states of other group members, and thus interactive life health management and social personal relationships between the group members may be further improved.

FIG. 2A illustrates a metaphorical image that visualizes life health information of a user, according to an embodiment of the present invention.

An example of the metaphor setting unit 120 may set a metaphor corresponding to life health information of the user, and the metaphor may be, for example, a flowerpot 200 in which a plant is planted. Here, a physical health state of the user, a psychological health state of the user, and interactive exchange information between the user and other people may be mapped onto a stem 210, a leaf 212, a flower 214, or insects such as a butterfly 218 and a dragon fly 216, to be expressed thereby.

For example, sizes of the stem 210 and the leaf 212 may indicate the physical health state of the user, denoting an amount of exercise of the user. Colors of the leaf 212 and the flower 214 may indicate the psychological health state of the user, denoting a stress level of the user. Also, a degree of activity of the butterfly 218 or the dragonfly 216 that fly around the flower 214 may indicate the degree of the social exchange state of the user.

FIG. 2B illustrates metaphorical images showing life health information of family members, according to an embodiment of the present invention.

For example, if the user of the life health information providing apparatus 100 or 160 is a mother, the personal life health information obtaining unit 110 may obtain life health information from the mother 260, a daughter 230, a son 240, a father 250, and a grandmother 270 for the mother to manage life health of the family members.

The metaphor setting unit 120 sets a metaphor with respect to life health information of all of the family members. The display unit 130 displays metaphorical images that are visualized for each of the family members on a life health information providing screen 220.

Accordingly, on the life health information providing screen 220 of the family members, a flower in a flowerpot 230 displaying life health information of the daughter, a flower in a flowerpot 240 displaying life health information of the son, a flower in a flowerpot 250 displaying life health information of the father, a flower in a flowerpot 260 displaying life health information of the mother, and a flower in a flowerpot 270 displaying life health information of the grandmother are displayed.

The mother may obtain life health information of each of the family members by seeing the conditions of the flowers in the flowerpots 230, 240, 250, 260, and 270. Also, the metaphor setting unit 120 may allow the user to change life health information that is mapped to each of metaphors for the protection of privacy. Accordingly, people other than the user may not recognize life health information of the family members by viewing the conditions of the flowers in the flowerpots 230, 240, 250, 260, and 270 of the life health information providing screen 220.

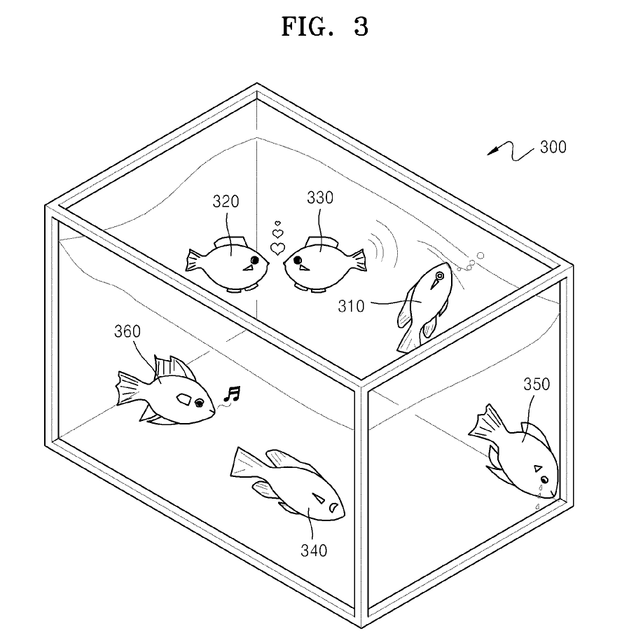

FIG. 3 illustrates metaphorical images of life health information of group members, according to another embodiment of the present invention.

The metaphor setting unit 120 may express metaphors of life health information of the family members with a plurality of fish in a fish bowl. Through the degree of activity of the fish, the behavior of the fish, and the affinity between the fish in the fishbowl, physical health states, psychological health states, and interactive exchange information between a person and other people among the family members may be expressed.

For example, first through sixth fish 310, 320, 330, 340, 350, and 360 are metaphorical images mapped in regard to a son, a mother, a father, a grandfather, a grandmother, and a grandson, respectively, among the family members. A metaphorical image of the first fish 310 floating on a water surface and not breathing normally may denote that the son is in a serious psychological state. A metaphorical image of the second fish 320 and the third fish 330 being on good terms denotes that mother and father are in conjugal harmony.

Also, a metaphorical image of the fourth fish 340 lying on the bottom of the fishbowl denotes that grandfather is sick. A metaphorical image of the fifth fish 350 playing alone at a corner of the fishbowl denotes a depressed state of the grandmother. A metaphorical image of the sixth fish 360 whistling a musical note denotes a happy and healthy state of the grandson.

Accordingly, the family members may detect life health states of other family members by using a metaphorical image 300 including the first through sixth fish 310, 320, 330, 340, 350, and 360 in regard to the life health information of all of the family members and may deal with the life health states of the family members accordingly, which enables easy management of the life and health of the family members.

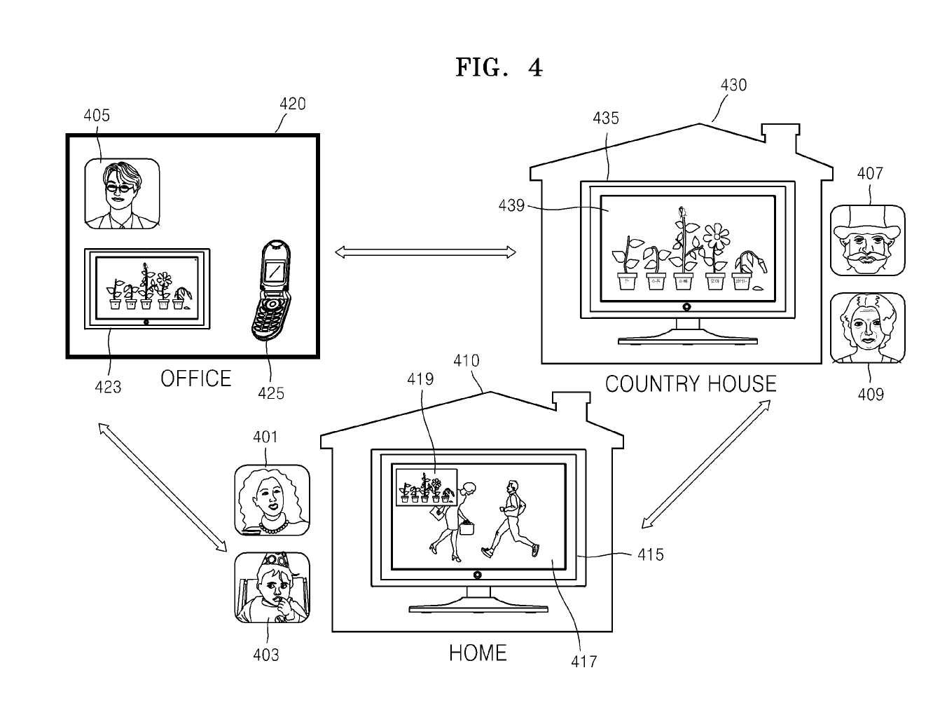

FIG. 4 illustrates a method of sharing life health information of family members, according to an embodiment of the present invention.

Metaphorical images of all of the family members may also be shared with family members who are a long distance away via a network. For example, it may be assumed that the life health information providing screen 220 of FIG. 2B is shared by a home 410, an office 420, and a country house 430.

At the home 410, a mother 401 and a son 403 may be watching a drama on a digital TV 415, and the life health information providing screen 220 of the family members may be displayed on a portion 419 of a main screen 417 in PIP (Picture-In-Picture) mode. In the office 420, a father 405 may see the life health information providing screen 220 of the family members by using a PDA 423 or a mobile phone 425. Also, in the country house 430, a grandfather 407 and a grandmother 409 may see the life health information providing screen 220 of the family members through a digital TV 435.

Accordingly, the family members may obtain life health information of other family members via a network by using the life health information providing apparatus 100 or 160, which is helpful to family members who are living a long distance from home.

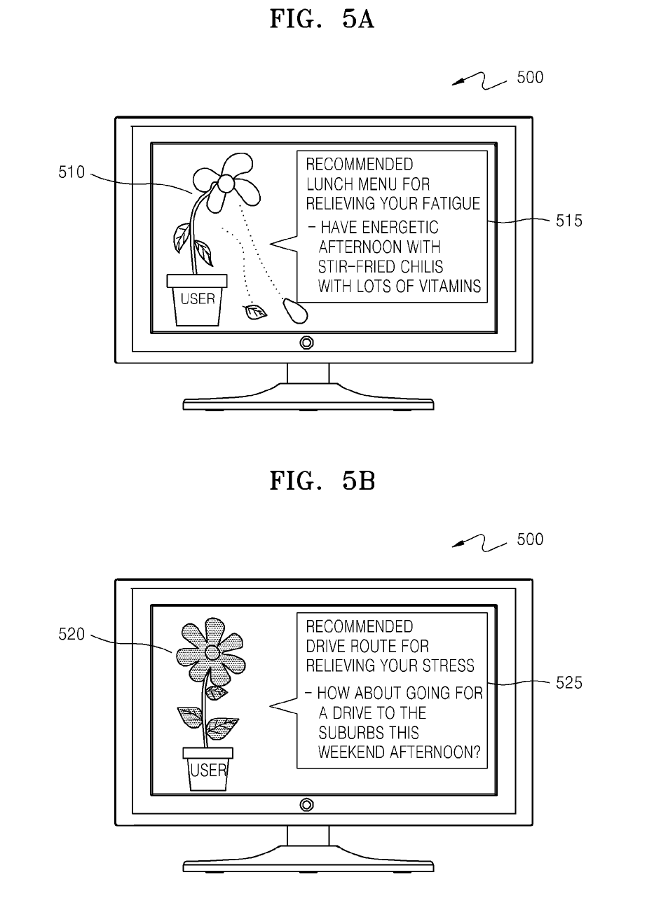

FIGS. 5A and 5B illustrate screens providing recommended life guiding plans with respect to life health information of the user, according to embodiments of the present invention.

Referring to FIG. 5A, a life health information providing screen 500 shows a flowerpot 510 having a flower and leaves that are either drooping or fallen. The flower in the flowerpot 510 indicates that physical fatigue of the user is great.

The recommended life guiding plan selecting unit 150 may determine that a physical lifestyle habit for relieving fatigue is needed for the user, based on information about a physical health state of the user obtained by using the personal life health information obtaining unit 110. The recommended life guiding plan selecting unit 150 may select a recommended diet for relieving fatigue by searching the life guiding plan database 140.

Accordingly, the display unit 130 may display recommended diet information 515 for relieving fatigue of the user as life guiding plan information on the life health information providing screen 500 of the user.

According to another embodiment, referring to FIG. 5B, the life health information providing screen 500 of the user displays a flowerpot 520 having a flower, a stem, and leaves that are colored black. The flower in the flowerpot 520 indicates that the user is psychologically stressed.

The recommended life guiding plan selecting unit 150 may determine that the user needs a stress relieving plan based on a psychological health state of the user that is obtained by using the personal life health information obtaining unit 110. The recommended life guiding plan selecting unit 150 may select a recommended route for a drive for relieving stress by searching the life guiding plan database 140.

Accordingly, the display unit 130 may display recommended drive route information 525 for relieving stress of the user as life guiding plan information on the life health information providing screen 500 of the user.

Here, only the recommended diet and the recommended drive route are described for convenience of description but other various physical lifestyle habitS, methods for stabilizing the mind, and methods of social exchange may also be recommended in consideration of the physical health state and the psychological health state of the user, and interactive exchange information between the user and other people by using the life health information providing apparatus 160.

For example, the life health information providing apparatus 160 may obtain personal log information about communications from a son to a father and communications from the father to the son from a mobile phone of the son. If it is determined that the frequency of communication between the son and the father is low, the life health information providing apparatus 160 may recommend a social exchange method for the son such as making a call to the father or sending a text message or an e-mail.

Accordingly, by using the life health information providing apparatus 160, current life health information of the user may be easily recognized and information about physical lifestyle habits, psychological life attitudes, and social exchange methods, which are needed to improve the life health of the user, may be obtained.

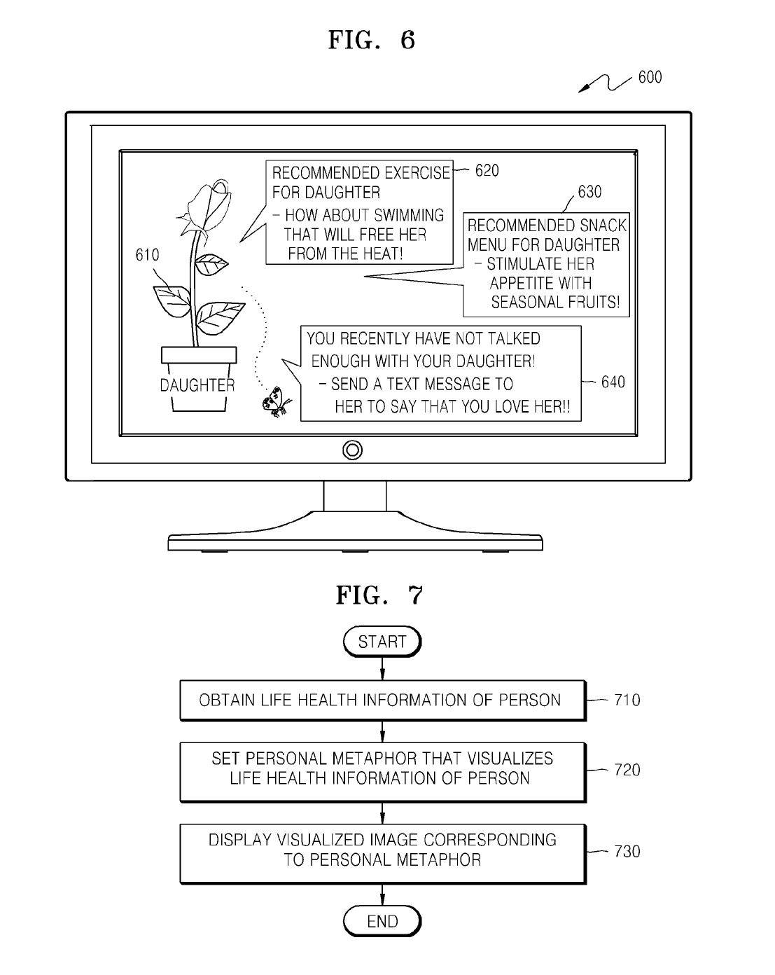

FIG. 6 illustrates an screen providing a recommended life guiding plan with respect to life health information of other people, according to an embodiment of the present invention.

By selecting a metaphorical image for a daughter from a life health information providing screen of the family members, a life health information screen 600 for the daughter may be displayed.

A flowerpot 610, in which a flowerpot having a flower that is not yet opened and small leaves drooping is planted, and a butterfly which is not actively moving around the flowerpot 610, are illustrated in the life health information screen 600 of the daughter. The plant may indicate that physical energy of the daughter is not sufficient, and the butterfly which is not active may indicate that a relationship between the daughter and her mother is estranged.

The recommended life guiding plan selecting unit 150 may determine that exercise and good diet are needed for the daughter to supplement her energy, based on the information about a physical health state of the daughter that is obtained by using the personal life health information obtaining unit 110. The recommended life guiding plan selecting unit 150 may select life guiding plan information in consideration of a current time and information about tendencies of the daughter that is obtained in advance.

Accordingly, after searching the life guiding plan database 140 and considering that the current season is summer and the daughter likes snacks, the recommended life guiding plan selecting unit 150 may select swimming and summer fruits as exercise and food, respectively, to supplement her energy in the summer.

Also, the recommended life guiding plan selecting unit 150 may determine that a communication method for improving the relationship between the daughter and her mother is needed, based on a social health state of the daughter obtained by using the personal life health information obtaining unit 110. Accordingly, the recommended life guiding plan selecting unit 150 may select ‘mother sends a text message to daughter’ as a communication method between the daughter and her mother for improving the relationship between the daughter and her mother.

Accordingly, the display unit 130 may display a recommended exercise 620 and a recommended snack 630 for the daughter to supplement her energy in the summer, and a communication method 640 for improving the relationship between the daughter and her mother on the life health information providing screen 600 of the daughter.

Accordingly, the user may easily recognize life health information of other people by seeing an intuitive metaphorical image by using the life health information providing apparatus 160 without any expert knowledge about health. Also, by using the life health information providing apparatus 160, the user may obtain information about physical lifestyle habits, psychological health states, and the interactive exchange information between people, which are needed for improving the life and health. Accordingly, not only the life and health of the user but that of other people may also be managed and good social relationships between the people may be maintained by using the life health information providing apparatus 160.

FIG. 7 is a flowchart illustrating a method of providing life health information, according to an embodiment of the present invention.

In step 710, life health information based on at least one of a physical health state of a person, a psychological health state of the person, and interactive exchange information between the person and other people is obtained. Here, personal life health information of not only a user but also other members of a group may be obtained.

In step 720, a personal metaphor that visualizes life health information of the person is set. Personal metaphorical images that intuitively visualize life health information of the user and other group members may be set. A metaphor regarding a social health state needs to be based on social exchange conditions between the person and other people.

In step 730, a visualized image corresponding to each of the personal metaphors is displayed. Also, a life health information providing screen of family members, on which personal metaphorical images of the family members are illustrated, may be shared between the family members over a long distance via a network.

In the method of providing life health information according to the current embodiment of the present invention, recommended life guiding plans for each individual may be searched for and provided as life health information. The life guiding plans may include diets for physical health, physical lifestyle habits such as doing exercise, methods of gaining psychological stability, and social exchange methods for social health.

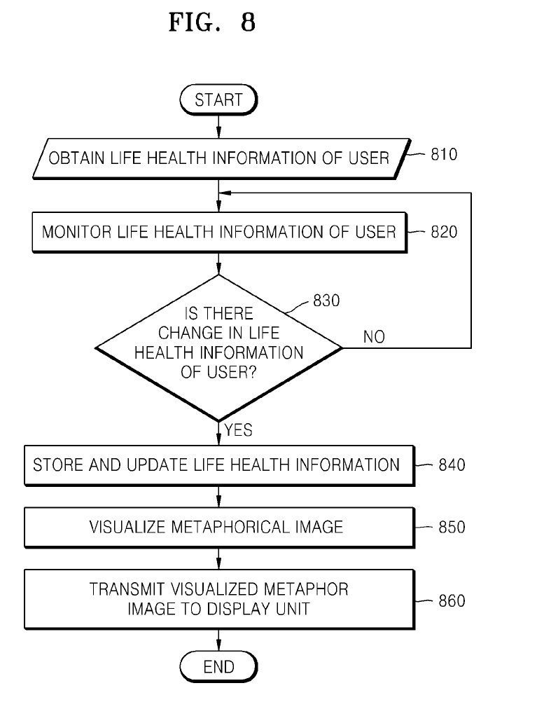

FIG. 8 is a flowchart illustrating a method of providing life health information, according to another embodiment of the present invention.

In step 810, life health information of a user is obtained, and the obtained life health information of the user is monitored in step 820. In step 830, if it is determined that there is no change in the life health information of the user, the process returns to step 820. If it is determined that there is a change in the life health information of the user in step 830, the changed life health information is stored and updated in step 840.

In step 850, a metaphorical image corresponding to the stored life health information is set, and a visualized metaphorical image is generated. In step 860, the visualized metaphorical image is transmitted to a display unit and displayed on the display unit.

According to the method of providing life health information according to the current embodiment of the present invention, not only personal physical and psychological health of a person but also the social health of the person based on social exchanges in social environments, are considered when managing the life and health of the person by using the life health information of the person. In addition, life guiding plans are suggested, thereby easily managing the life and health of the person. Also, since life health information of each group member of a group and information about life guiding plans for each of them may be shared between all the group members, interactive life health management between the group members and social relationships therebetween may be further improved.

The embodiments of the present invention can be written as computer programs and can be implemented in general-use digital computers that execute the programs using a computer readable recording medium. Examples of the computer readable recording medium include read-only memory (ROM), random-access memory (RAM), CD-ROMs, magnetic tapes, floppy disks, optical data storage devices, etc. (e.g., transmission through the Internet).

While the present invention has been particularly shown and described with reference to exemplary embodiments thereof, it will be understood by those of ordinary skill in the art that various changes in form and details may be made therein without departing from the spirit and scope of the present invention as defined by the following claims. The embodiments should be considered in a descriptive sense only and not for purposes of limitation. Therefore, the scope of the invention is defined not by the detailed description of the invention but by the appended claims, and all differences within the scope will be construed as being included in the present invention.

What is claimed is:

1. A method of providing life health information, the method comprising:
obtaining life health information based on at least one of a physical health state of a person, a psychological health state of the person, and interactive exchange information between the person and other people;
setting a personal metaphor that visualizes the life health information of the person; and
displaying a visualized image corresponding to the personal metaphor.

2. The method of claim 1, wherein the person is one member of a group, wherein a user and the other people are included in the group.

3. The method of claim 1, wherein obtaining the life health information comprises obtaining personal log information about communications between the person and the other people, which is stored in a communication device of the person as life health information, based on the interactive exchange information between the person and the other people.

4. The method of claim 1, wherein obtaining the life health information comprises obtaining physical health index information measured using a physical health measuring device of the person as life health information based on the physical health state.

5. The method of claim 1, wherein obtaining the life health information comprises obtaining resultant information that is measured using a device for measuring psychological stability of the person as life health information based on the psychological health state.

6. The method of claim 1, wherein in setting the personal metaphor, the personal metaphor with respect to the life health information based on the interactive exchange information between the person and the other people is expressed by considering a combination of a degree of communication from the person to the other people and a degree of communication from the other people to the person.

7. The method of claim 1, wherein in setting the personal metaphor, the personal metaphor with respect to the life health information based on the interactive exchange information between the person and the other people separately expresses a degree of communication from the person to the other people and a degree of communication from the other people to the person.

8. The method of claim 1, wherein in setting the personal metaphor, the personal metaphor corresponding to the life health information of the person is set to include a first sub-metaphor indicating the physical health state of the person, a second sub-metaphor indicating the psychological health state of the person, and a third sub-metaphor indicating the interactive exchange information between the person and the other people.

9. The method of claim 1, wherein in setting the personal metaphor, a first sub-metaphor indicating the physical health state of the person, a second sub-metaphor indicating the psychological health state of the person, and a third sub-metaphor indicating the interactive exchange information between the person and the other people are separately expressed.

10. The method of claim 1, further comprising providing information about a recommended life guiding plan to the person based on the obtained life health information of the person.

11. The method of claim 10, wherein the information about the recommended life guiding plan includes information about a physical lifestyle habit including at least one of a diet and an exercising method that are selected in consideration of the physical health state of the person.

12. The method of claim 10, wherein the information about the recommended life guiding plan includes information about a method of gaining psychological stability including methods for relieving stress or hobbies that are selected in consideration of the psychological health state of the person.

13. The method of claim 10, wherein the information about the recommended life guiding plan includes information about social exchange activity including methods of contacting other people or activities that may be shared with the other people that are selected in consideration of the interactive exchange information between the person and the other people.

14. The method of claim 2, further comprising sharing life health information of group members between the group members by transmitting visualized images corresponding to personal metaphors of the group members.

15. The method of claim 3, wherein the personal log information about communications between the person and the other people includes at least one of email exchange log information, telephone exchange log information, text message exchange log information, chatting log information between the person and the other people, and information about recorded information of appointments with the other people in schedule data of the person.

16. A life health information providing apparatus comprising:
a personal life health information obtaining unit obtaining life health information based on at least one of a physical health state of a person, a psychological health state of the person, and interactive exchange information between the person and other people;
a metaphor setting unit setting a personal metaphor that visualizes the life health information of the person; and
a display unit displaying a visualized image corresponding to the personal metaphor.

17. The life health information providing apparatus of claim 16, wherein the person is one member of a group, wherein a user and the other people are included in the group.

18. The life health information providing apparatus of claim 16, wherein the personal life health information obtaining unit further obtains life health information of the other people from the other people via a network.

19. The life health information providing apparatus of claim 16, further comprising:
a life guiding plan database that stores information about a recommended life guiding plan that is to be provided to the person based on the life health information of the person; and
a recommended life guiding plan selecting unit that selects information about the recommended life guiding plan for the person based on the obtained life health information of the person,
wherein the display unit displays the information about the selected recommended life guiding plan.

20. A computer readable recording medium having embodied thereon a program for executing the method of claim 1.
